(19) United States
(12) Patent Application Publication
(10) Pub. No.: US 2010/0227772 A1
(43) Pub. Date: Sep. 9, 2010

(54) DETERMINING THE INTERACTION BETWEEN NUCLEIC ACIDS AND NUCLEIC ACID BINDING MOLECULES

(75) Inventors: Walter Gehring, Therwil (CH); Rudolf Rigler, St. Sulpice (CH); Stefan Wennmalm, Johanneshov (SE); Paul Baumgartner, Reinach (CH); Marina Hugot-Beaufils, Bartenheim (FR)

Correspondence Address: ROTHWELL, FIGG, ERNST & MANBECK, P.C., 1425 K STREET, N.W., SUITE 800, WASHINGTON, DC, 20005, US

(73) Assignee: University of Basel

(21) Appl. No.: 12/515,317

(22) PCT Filed: Nov. 16, 2007
(86) PCT No.: PCT/IB07/03523

§ 371 Date: May 11, 2010

(30) Foreign Application Priority Data
Nov. 17, 2006 (GB) 0624055.0

(51) Int. Cl. C40B 30/04; G01N 33/00

(52) U.S. Cl. 506/9; 436/94

(57) ABSTRACT

The present invention refers to methods for determining the interaction between a nucleic acid molecule and a nucleic acid binding molecule. The methods of the invention are particularly suitable for the analysis of genes associated with pathologic disorders and for the identification of novel therapeutic agents.

The present invention refers to methods for determining the interaction between a nucleic acid molecule and a nucleic acid binding molecule. The methods of the invention are particularly suitable for the analysis of genes associated with pathologic disorders and for the identification of novel therapeutic agents.

Interactions between nucleic acids and nucleic acid binding molecules such as proteins are generally studied by footprint and gel shift assays or similar methods, which are not amenable to genomic screening. Further, these methods are usually carried out in a two-phase system in thus the kinetics of interaction cannot be easily interpreted and/or are not suitable for a genomic approach either.

According to the present invention, it was found that interactions between nucleic acids and nucleic acid binding molecules can be analysed by fluorescence-based methods, wherein the diffusion time of the free nucleic acid binding molecule as compared to that of the bound molecule is determined. These measurements are fast, they can be automated e.g. in a microtiter format and they can be used for high throughput screening. Further, the method allows the analysis of large nucleic acid molecules e.g. chromosomal segments.

Doi et al. (Genome Res. 12 (2002), 487-492) describe fluorescence labelling and high-throughput assay technologies for in vitro analysis of protein interactions. The binding of fluorescent-labeled Fos and Jun proteins to short (20 nucleotide) fluorescence-labelled double-stranded DNA molecules was measured by Fluorescence Cross-Correlation Spectrometry (FCCS). The feasibility of FCCS measurements of the interaction between nucleic acid binding molecules and longer nucleic acid molecules is, however, neither disclosed, nor suggested.

Thus, the present invention relates to a method for determining the interaction between a nucleic acid molecule and a nucleic acid-binding molecule, comprising the steps:
(a) providing a nucleic acid-binding molecule which carries a first fluorescent labelling group,
(b) contacting the nucleic acid-binding molecule with a nucleic acid molecule having a length of more than 100 nucleotides, and
(c) measuring the diffusion time of the nucleic acid-binding molecule in solution and thereby determining the interaction of the nucleic acid-binding molecule and the nucleic acid molecule.

The nucleic acid binding molecule may be any molecule which is capable of binding to a nucleic acid and from which the diffusion time can be measured in solution such as a protein, a peptide, an aptamer, a further nucleic acid, e.g. an RNA molecule or a low molecular weight compound, e.g. a compound having a molecular weight of about 2500 Da or less. Preferably, the nucleic acid binding molecule is a transcription factor or another gene regulatory molecule, i.e. a molecule which binds to a nucleic acid, preferably in or adjacent to a transcriptional control sequence and thereby modulates, e.g. stimulates or inhibits transcription. Preferred examples of transcription factors are proteins such as helix-turn-helix molecules, e.g. homeobox proteins or other transcription factors such as zinc finger molecules, leucin zipper molecules, hormone receptors etc., microRNAs or RNA protein complexes.

Preferably, the nucleic acid binding molecule binds sequence-specifically to the nucleic acid molecule. Further, it is preferred that the binding does not involve hybridization, particularly not double-strand formation by hybridization, e.g. DNA-DNA, or RNA-DNA or RNA-RNA double-strand formation.

The nucleic acid binding molecule carries one or several fluorescent labelling groups. The fluorescent labelling groups may be a low molecular weight compound, e.g. a compound with a molecular weight of about 2500 Da or less such as fluorescein, rhodamine or cyanine dyes. Especially preferred dyes are Bodipy-630, Bodipy-650, CY3, CY5 or Flash. These fluorescent dyes may be coupled to the nucleic acid binding molecule according to standard methods, e.g. by using a linker.

In a further embodiment, the fluorescent labelling group may be a fluorescence protein, e.g. a green fluorescence protein (GFP) including variants thereof. For example, the nucleic acid binding molecule may be a fusion protein comprising a first nucleic acid binding domain and a second fluorescent domain.

The nucleic acid molecule may be selected from double-stranded DNA molecules, single-stranded DNA molecules, RNA molecules and nucleic acid analogues. Preferably, the nucleic acid molecule is double-stranded DNA. The nucleic acid molecule may have any length which allows efficient binding of the nucleic acid binding molecule, e.g. up to 10000 nucleotides or more. Preferably, the nucleic acid molecule is a long molecule with a length of from more than 100 nucleotides, e.g. from about 500 to about 10000 nucleotides, more preferably from about 1000 to about 5000 nucleotides. Preferably, the nucleic acid molecule has a length of about 500 nucleotides or more.

The molecular weight of the nucleic acid binding molecule (including the fluorescent group) is preferably up to 50%, more preferably up to 25% and most preferably up to 10% of the molecular weight of the nucleic acid molecule. Thus, a sufficient molecular weight difference between the free and the bound nucleic acid binding molecule is provided which results in a significant difference between the diffusion time of the free and the bound molecule.

The nucleic acid molecule may be unlabelled or may carry a second fluorescent labelling group which is different from the first fluorescent labelling group. For example, the second fluorescent group may be a low molecular weight compound or a protein as described above. The nucleic acid molecule may carry the second fluorescent labelling group at its 5′ end and/or at its 3′ end. The coupling of fluorescent labelling groups to nucleic acid molecules may be carried out according to known standard methods, e.g. using linkers. Preferably, the nucleic acid molecules are labelled by enzymatic methods, e.g. by adding fluorescent labelled nucleotides to the 3′-ends of nucleic acid fragments by Terminal Transferase, or by chemical methods.

The diffusion time of the nucleic acid binding molecule is measured in solution, i.e. the nucleic acid binding molecule, the nucleic acid molecule and the complex between nucleic acid and nucleic acid binding molecule are not bound to a solid support and/or entrapped in a gel. The invention is based on the finding that the diffusion time of the labelled nucleic acid binding molecule increases upon binding to the nucleic acid molecule, which can be detected. Since the measurements are carried out in solution, even kinetic parameters, such as the on-rates, the off-rates, the dissociation rate constant (kdiss) and/or the dissociation time (tdiss) can be determined. Further, the inhibition constant (ki) of test compounds can be determined.

Preferably, the diffusion time of the nucleic acid binding molecule is measured by single-molecule detection, wherein the concentration of the nucleic acid binding molecule and/or the nucleic acid are less than 10⁻⁸ mol/l, preferably e.g. about 10⁻⁸ to about 10⁻¹¹ mol/l. Thus, compared to other methods, minimal amounts of nucleic acid and nucleic acid binding molecules are consumed.

For example, the detection can be performed by means of confocal single molecule detection, such as fluorescence-correlation spectroscopy (FCS), whereby a very small, preferably confocal volume element of the sample is exposed to the exciting light of a laser, exciting the fluorescence labels present in this measure volume to emit fluorescent light and wherein the fluorescence radiation emitted from the measuring volume is determined by means of a photodetector. Based on the different diffusion characteristics of the free and bound nucleic acid binding molecule, a correlation between the time-related changes of the measured emission and the presence of a labelled molecule is established, so that single molecules in the measuring volume can be identified. With regard to the details of performing this process and details of the apparatus used in the detection process it is referred to Rigler et al. (Eur. Biophys. J. with Biophys. Lett. 22 (1993), 169-175 and confocal single molecule determination has also been described by Rigler and Mets (Soc. Photo-Opt. Instrum. Eng. 1921 (1993), 239 et seq.) and Mets and Rigler (J. Fluoresc. 4 4 (1994), 259-264).

Alternatively, or rather additionally, the detection can also be performed by means of a time-resolved decay measurement, a so-called time gating, such as described by Rigler et al., “Picosecond Single Photon Fluorescence Spectroscopy of Nucleic Acids”, in: “Ultrafast phenomena”, D. H. Auston, Ed. Springer 1984. In this context the excitation of the fluorescence molecules is brought about within a measure volume and subsequently—preferably after a period of ≧100 ps—an opening of a detection interval at the photodetector. In this manner background signals created by Raman-effects can be kept at a sufficiently low level, in order to render possible an essentially undisturbed detection.

When the nucleic acid molecule carries a second fluorescent labelling group, the detection can be performed by means of fluorescence cross-correlation spectroscopy FCCS, which is e.g. described by Schwille et al. (Biophys. J. 72 (1997), 1878-1886), Rigler et al. (J. Biotechnol. 63 (1998), 97-109) or Kettling et al. (Proc. Natl. Acad. Sci. USA 95 (1998), 1416-1420). The second fluorescent labelling group may be selected from fluorescent labelling groups as described above provided that it is different from the first fluorescent labelling group in at least one fluorescence parameter, e.g. emission wave length and/or fluorescence decay time.

The confocal detection volume is preferably about 0.01 fl to 100 pl, preferably about 0.1-100 fl and more preferably about 0.1-1 fl. The actual detection volume for a specific test system may be determined by calibration, i.e. based on the diffusion coefficients of rodlike molecules as described in Tirrado et al. (J. Chem. Phys. 81 (1984), 2047-2052).

The fluorescence parameters, e.g. the intensity of the fluorescent labelling groups of the nucleic acid binding molecule and optionally of the nucleic acid molecule may considerably change upon complex formation caused by fluorescence quenching and/or fluorescence resonance energy transfer (FRET). Thus, it is preferred to carry out a correction of the fluorescence intensity for the free and/or bound nucleic acid binding molecule and optionally for the free and/or bound nucleic acid molecule (if it carries a fluorescent labelling group). Preferably, the correction comprises the calculation of autocorrelation amplitudes as described in the example in detail.

The reaction is preferably carried out on a carrier, e.g. on a microfluidic carrier or a microtiter plate. In the method of the invention, a plurality of measurements may be carried out in parallel. For example, a plurality of measurements may be carried out in different detection volumes of a single sample and/or in detection volumes of different separate samples. The parallel measurements may be carried out on an array comprising a plurality of different determination sites located on a single carrier, e.g. on a microtiter plate or a microfluidic array or on any other suitable array.

The fluorescence measurement comprises irradiation of the sample with a suitable light source, e.g. a laser or a plurality of lasers suitable for exciting the fluorescence of the labelling groups, in at least one measuring volume. The emitted fluorescence radiation may be detected using suitable optical systems, e.g. fluorescence detectors such as avalanche photodiodes or CCD detection matrices. Preferably, the method is carried out as an automated procedure.

In a first preferred embodiment, a plurality of DNA molecules, e.g. “tiled” DNA molecules which have short overlaps at both ends can be analysed for the presence of binding sites for nucleic acid binding molecules such as transcription factors or other gene regulatory proteins of interest. In this embodiment, the DNA molecules are preferably derived from genomic DNA, e.g. human genomic DNA and/or have a length of at least 500, more preferably at least 1000 nucleotides. This procedure allows a rapid identification of DNA target sites and target genes in a representative portion is of a genome, e.g. the human genome.

In a further preferred embodiment, the binding of the nucleic acid binding molecule to its target site may be measured in the presence of a test compound which might interfere with the binding. For example, the test compound may be obtained from libraries of chemical molecules, aptamers or peptides. Determining the binding constants and/or kinetics in the presence and absence of the test compound will lead to the identification of novel therapeutic agents which may act as antagonists or agonists of a nucleic acid binding molecule, e.g. a transcription factor. Since the nucleic acid binding molecule may either be a repressor or an activator of transcription, the novel identified agent may either repress or derepress the expression of the respective target gene. In this embodiment, it is preferred to carry out a high throughput screening procedure involving the analysis of a plurality of test compounds.

Further, the present invention is explained in more detail by the following Figures and Examples.

### FIGURE LEGENDS

FIG. 1. Assay strategy for screening interactions between transcription factors (here the protein antennapedia homeodomain (AntpHD)) and nucleic acids. (A) Various methods to specifically label the transcription factor: i) using the Cys-Cys-Pro-Gly-Cys-Cys genetic tag with a biarsenical fluorescein derivate called FlAsH, ii) using a conjugate with a fluorescent protein (FP), iii) using a dye covalently attached to the protein, or iv) using quantum dots (QD). (B) Cloning strategy for construction of an Antp homeodomain expression plasmid pAop3. Only the restriction site BamHI important for the cloning was represented. The Antp homeodomain contains the amino acids residues 297-364. The homeobox and homeodomain (defining the homeobox as 180 bases and as 60 amino acids, respectively) are boxed in. The amino acids outside the homeodomain representing the tetracystein motif are given in single. (C) Schematic showing the binding of the fluorescently labeled protein to the DNA.

FIG. 2. Saturation curve of Antp HD-GFP and HB1 (36 bp) measured by FCCS. For this curve K_{D}=6.3 nM. The fractional saturation is plotted against the free DNA concentration (Ngr/Ng means the number of green-red-molecules divided by the number of green molecules).

FIG. 3. Saturation curve of Antp HD-GFP and BS2 (16 bp) measured by FCCS. For this curve K_{D}=8.6 nM.

### EXAMPLE

### DNA-Binding Studies of the Antennapedia Homeodomain by Fluorescence Cross-Correlation Spectroscopy

### 1. Summary

Hox transcription factors regulate a large number of target genes, but so far very few of these have been identified. Since the consensus target sequences recognized by homeodomain proteins are rather loosely defined, bioinformatics gives a large number of false positive sequences. Therefore, bioinformatic analysis has to be complemented by DNA binding studies in vitro and in vivo, and finally by functional genetics.

A new approach for the genome-wide detection of DNA-binding sites in vitro involves the application of Fluorescence Correlation Spectroscopy (FCS). As a model system, synthetic homeotic genes, of two similar, but functionally distinct Hox genes, Antennapedia (Antp) and Sex combs reduced (Scr) are used. These synthetic genes encode relatively short peptides consisting of the YPWM motif and the homeodomain fused to the Green Fluorescent Protein (GFP). These synthetic genes are biologically active when tested in transgenic flies and give 100% homeotic transformations when driven by an appropriate enhancer. This allows us to extend our studies to the in vivo situation.

Using FCS pilot experiments with the Antp-Homeodomain (HD) carried show that DNA fragments as large 4 kb can be used to detect specific HD binding sites and to determine the affinity constants which for known DNA target sites are in the nanomolar range. Further experiments encompass the tiling of large regions of the Drosophila genome into microtiter plates, in pieces of approx. 1 kb and to measure binding of the GFP-tagged transcription factor to the DNA in each well which will take only a few seconds. For the detection of DNA binding it is sufficient to label only the protein partner with GFP, and to measure the increased diffusion time when the DNA-protein complex forms. In this way we can screen a large number of unlabelled DNA fragments very rapidly. For a more in depth analysis the DNA can also be labeled, e.g. with Bodipy 630/650. An automatisation is possible.

Our pilot study was carried out with Antp, but we are also planing to use Scr, which is the master control gene for salivary gland formation. This will allow us to examine DNA binding in vivo, in living salivary gland giant polytene chromosomes by in vivo imaging techniques.

### 2. Detailed Description of Experiments

Homeotic proteins serve as transcription factors that control a large number of subordinate genes involved in morphogenesis. Gain-of-function mutations in Antennapedia (Antp) lead to homeotic transformation of the antennae into second legs, whereas loss-of-function of Antp leads to a transformation in the opposite direction from the second thoracic segment into antennal and head structures which indicates that Antp specifies the second thoracic segment (T2). Sex combs reduced (Scr) specifies the first thoracic (T1) and labial segment (Lb). Ectopic expression of Scr in more posterior segments leads to the formation of a second pair of salivary glands which contain giant polytene chromosomes. Both of these Hox genes contain a homeobox, which represents the DNA binding domain of these transcription factors. The Antp homeodomain (HD) has previously been found to form a very stable complex with its target DNA with a K_{D} in the nanomolar range (Affolter M., Percival-Smith A., Müller M., Leupin W. and Gehring W J., (1990). DNA binding properties of the purified Antennapedia homeodomain. Proc. Natl. Acad. Sci. USA 87, 4093-4097). This study was carried out by gel mobility shift assays, which have their limitations.

For a more detailed binding study, we have begun to use Fluorescence Cross-Correlation Spectrometry (FCCS). In FCCS, with the use of two lasers with different wavelength, correlated movement of molecules carrying different fluorescent labels is revealed.

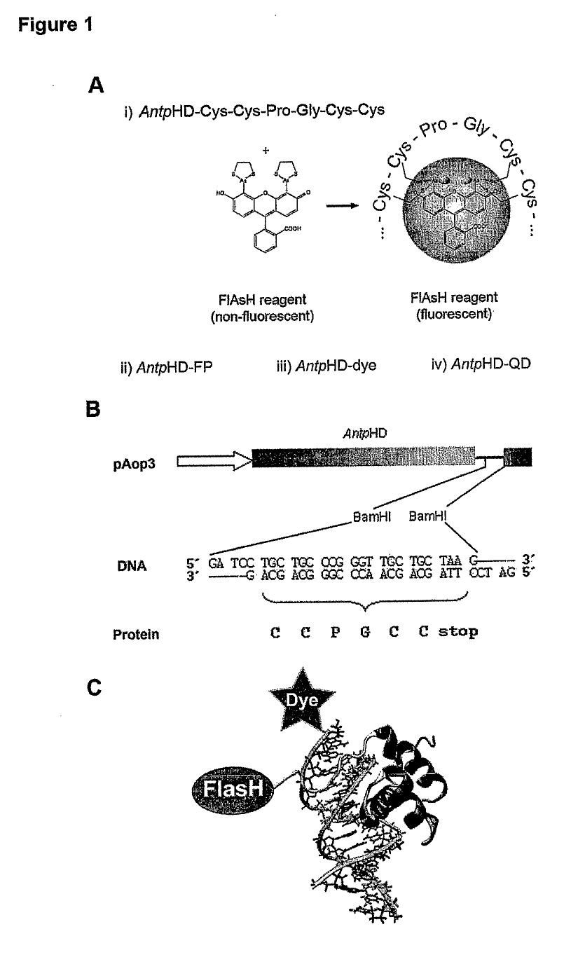

In FIG. 1 a preferred embodiment of an assay strategy for screening interactions between transcription factors and nucleic acids is shown.

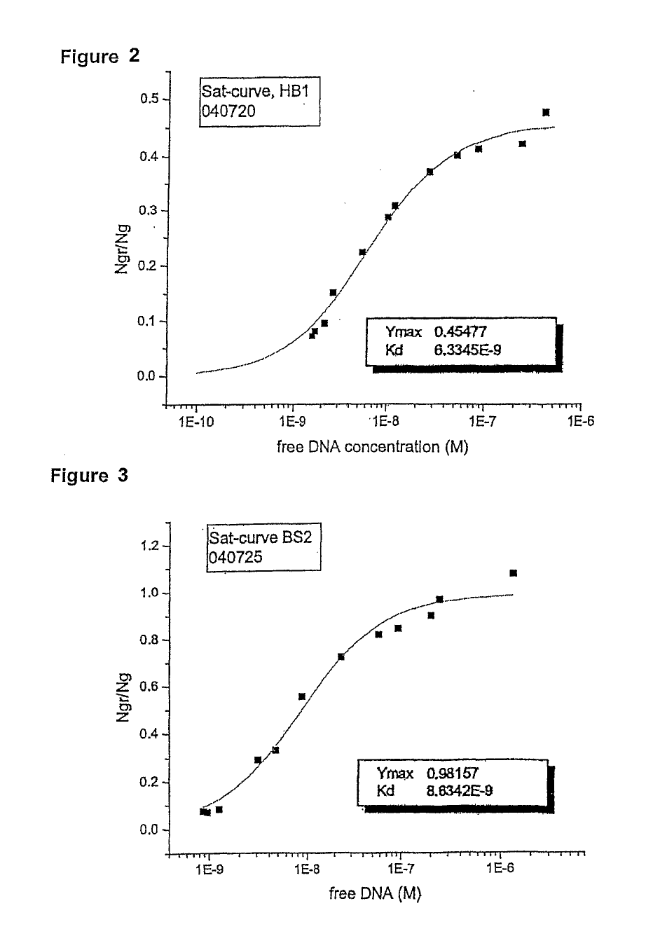

We tagged the YPWM motif plus homeodomain (HD) of Antp with Green Fluorescent Protein and two known DNA target sites (BS2 and HB1) were labeled with Blodipy 630/650. First, we determined the saturation curves for HB-1 (FIG. 2) and for BS-2 (FIG. 3). HB1 is a target for the Ultrabithorax (Ubx) protein, but it also binds strongly to other HD-proteins, such as Antp-HD.

The curves in FIGS. 2 and 3 are only two examples of the saturation curves that have been measured. So far 10 saturation curves with HB1-36 and 3 with BS2-16 have been performed and analyzed. The average K_{D} for BS2 so far is 5.1 nM and for HB1 the average K_{D} is 3.6 nM. The difference between the two is expected; since HB1 contains three binding sites for HD but BS2 only contains one binding site, the affinity for HB1 should be stronger and to thus K_{D} should be lower.

The measured K_{D} values are in reasonable agreement with our previous studies; but in our previous study we have largely overestimated the half-life of the DNA-protein complex.

1. A method for determining the interaction between a nucleic acid molecule and a nucleic acid-binding molecule, comprising the steps:
(a) providing a nucleic acid-binding molecule which carries a first fluorescent labelling group,
(b) contacting the nucleic acid-binding molecule with a nucleic acid molecule having a length of more than 100 nucleotides which carries a second fluorescent labelling group which is different from the first fluorescence labelling group, and
(c) measuring the diffusion time of the nucleic acid-binding molecule in solution by single molecule detection via Fluorescence Cross-Correlation Spectrometry (FCCS) and thereby determining the interaction of the nucleic acid-binding molecule and the nucleic acid molecule.

2. The method of claim 1, wherein the nucleic acid-binding molecule is selected from proteins, peptides, aptamers, nucleic acids and low molecular weight compounds.

3. The method of claim 1, wherein the nucleic acid-binding molecule is a transcription factor.

4. The method of claim 3, wherein the transcription factor is selected from proteins, RNAs, such as micro-RNAs, and protein-RNA complexes.

5. The method of claim 1, wherein the fluorescent labelling group is a low molecular weight compound.

6. The method of claim 1, wherein the fluorescent labelling group is a protein, preferably a Green Fluorescence Protein (GFP).

7. The method of claim 1, wherein the nucleic acid molecule is selected from double-stranded DNA molecules, single-stranded DNA molecules, RNA molecules and nucleic acid analogues.

8. The method of claim 1, wherein the nucleic acid molecule has a length of up to 10 000 nucleotides, preferably from about 1000-5000 nucleotides.

9. The method of claim 1, wherein the nucleic acid molecule carries the second fluorescent labelling group at its 5′-end and/or its 3′-end.

10. The method of claim 1, wherein the single molecule detection is carried out in a confocal detection volume.

11. The method of claim 10, wherein the confocal detection volume is about 0.01 fl-100 pl, preferably about 0.1-100 fl, more preferably about 0.1-1 fl.

12. The method of claim 10, wherein the detection volume is determined by calibration.

13. The method of claim 1, wherein a correction of the fluorescence intensity for the free and/or bound nucleic acid-binding molecule is carried out.

14. The method of claim 1, wherein a plurality of measurements is carried out in parallel.

15. The method of claim 14, wherein the parallel measurements are carried out on an array format.

16. The method of claim 1, which is an automated procedure.

17. The method of claim 1, wherein a plurality of overlapping DNA molecules derived from genomic DNA is analysed for the presence of binding sites for a nucleic acid-binding molecule.

18. The method of claim 1, wherein the interaction between the nucleic acid molecule and the nucleic acid-binding molecule is determined in the presence of a test compound.

19. The method of claim 1, wherein the interaction between the nucleic acid molecule and the nucleic acid binding molecule is determined in the presence of a test compound and where the inhibition constant (ki), the dissociation rate constant (kdiss) and/or the dissociation time (tdiss) are determined.

20. The method of claim 1, which is a high throughput screening procedure.
